(19) United States
(12) Patent Application Publication
(10) Pub. No.: US 2004/0071589 A1
(43) Pub. Date: Apr. 15, 2004

(54) Odor control through air-facilitated injection of hydroxyl radicals

(76) Inventors: Guy G. Morneault, Kelowna (CA); Stephen G. Morneault, Kelowna (CA)

Correspondence Address: BENNETT JONES, C/O MS ROSEANN CALDWELL, 4500 BANKERS HALL EAST, 855 - 2ND STREET, SW, CALGARY, AB, T2P 4K7, CA

(21) Appl. No.: 10/671,837

(22) Filed: Sep. 29, 2003

Related U.S. Application Data
(60) Provisional application No. 60/416,913, filed on Oct. 9, 2002.

(30) Foreign Application Priority Data
Jan. 24, 2003 (CA) 2,417,346

(51) Int. Cl. A61L 9/015; A61L 9/20

(52) U.S. Cl. 422/4; 422/24; 422/29; 422/121

(57) ABSTRACT

An apparatus for decontaminating air within an enclosed workspace located downstream and in fluid communication with the apparatus. The apparatus includes a housing containing an array of ultraviolet lamps mounted within an enclosure in the housing. The enclosure has an intake aperture and an exhaust aperture. The housing and the array form an airflow processor such that uncontaminated air entering the intake aperture passes through the array before exiting the exhaust aperture. An airflow motivator, which may be a fan, urges the airflow through the housing and the array from the intake aperture and out through the exhaust aperture. A downstream conduit, which may be a flexible hose, is in fluid communication between the exhaust aperture and the workplace directs the airflow into the workplace after being processed in the airflow processor. The intake aperture is positionable relative to the workspace so that the airflow entering the intake aperture is uncontaminated air.

### CROSS REFERENCE TO RELATED APPLICATION

The application claims priority from U.S. Provisional Patent Application No. 60/416,913, filed Oct. 9, 2002 and from Canadian Patent Application No. 2,417,346, filed Jan. 24, 2003.

### FIELD OF THE INVENTION

This invention relates to a process and apparatus to decontaminate volumes of air and achieve odor control, such as elimination of sulphuric fumes produced at wastewater treatment plants, the corresponding lift stations and collection systems, as well as in other industrial and commercial work sites.

### BACKGROUND OF THE INVENTION

One way to dispose of noxious gasses emitted by industrial manufacturing processes and in the treatment and disposal of organic and inorganic wastes is simply to allow the fumes to vent freely into the atmosphere. However, increasingly, environmental regulations and the weight of public opinion in opposition to the practice require the proper elimination of such offensive odors. Conventional odor elimination includes the use of wet-scrubbing, a technique by which odorous gasses are funneled into a treatment vessel and treated by a chemical mist or shower prior to the release of the treated air stream into the atmosphere. Another conventional technology involves the use of biological media filters, which are large containment structures built above-ground or underground, and filled with layers of various naturally absorbing materials, such as bark mulches, etc., and particular bacteria cultures. A contaminated air stream is collected and piped into the biofilter, with the air then finding its way out, now odorless, into the atmosphere.

Another conventional method in use, in particular in smaller sites, is the utilization of various kinds of activated charcoal. Fumes are channeled through a vessel containing a fresh load of charcoal, which by adsorption removes a range of volatile compounds from the air stream, rendering it odorless.

The application of ozone, usually generated from the electric-arc ionization of either ambient oxygen, or with the aid of pure oxygen, is also found in the prior art, either as a stand-alone technique or as a complement to others, for example in tandem with the use of activated charcoal. Ozonation in the prior art then is the oxidization of odorous gas molecules into simpler, less noxious and less odorous forms. Its use however has cost and health safety drawbacks. In the prior art, ozonation of closed chambers, such as the wet wells of lift stations, has been tried. High concentrations of ozone are generated to flood, virtually by force of gravity, the air portion of the chamber as the ozone is produced. Air movement has to be minimal so as to not disturb the electric arc within the generator. Workmen should not be present in the chamber due to the high concentration of ozone in the air.

Also in prior art, a different technological approach, photocatalytic oxidation has been tried. Photocatalytic oxidation is a process of gas oxidization that utilizes photon energy emitted from ultraviolet (alternatively referred to herein as UV) light lamps, either of the low pressure or of the medium pressure variety, operating at wavelengths ranging roughly between 170 and 255 nanometers, but favoring the former end (also known as the UV-Vacuum range). Photon energy catalyses a chain of photochemical reactions that lead to four contemporaneous processes: the photolysis of oxygen in the form of O₂; the photolysis of the oxidizing agent O₃ (ozone) formed during the UV irradiation of oxygen; the photolysis of water molecules (H₂O); and the photolysis of pollutant molecules and decay products. The ozone that is produced during the first photolytic reaction, the photolysis of oxygen, occurs with the intervention of UV light at the 185 nm frequency. This ozone acts then more as a catalyst for further reactions than as an oxidant itself, and ultimately most remaining ozone is degraded back to stable O₂ by the action of UV light at 254 nm. The intervening ozone in tandem with the photolysis of water molecules in the ambient air together lead to the production of hydroxyl radicals (OH—) thusly: O+H₂O→OH— and O₃+H₂O+hᵥ→O₂+H₂O₂ followed by H₂O₂+hᵥ→OH— (where hᵥ stands for the energy from UV light).

Hydroxyl radicals are highly efficient oxidants, more so than ozone, and react more quickly with volatile molecules. The molecules of contaminant gases that make up nuisance odors (including a broad range of compounds such as hydrocarbons or VOCs, solvents, ammonia, sulphurous and chlorinated compounds) are degraded in two ways: First, contaminated gases are degraded directly by the action of UV photons, to the extent that their particular UV absorption coefficient allows as different gasses break down at different wavelength and energy levels. Second, contaminant gases are degraded under the oxidizing effect of the hydroxyl radicals, which break down the odorous compounds by attacking the molecular bonds, starting with double bonds. Each breakdown process itself releases a number of OH— active radicals, which in turn go to work on the next degrading reaction, in a self-reinforcing mechanism that ultimately leads to the complete mineralization of the pollutant molecules into carbon dioxide, water vapor and mineral acids or elemental forms of sulfur, chlorine and nitrogen.

A variation of photocatalytic oxidation, dubbed Advanced Photocatalytic Oxidation (APO) has been also applied. It is defined by the complementary utilization of any of ozone, hydrogen peroxide H₂O₂ or reactive material surfaces such as titanium dioxide TiO₂ in tandem with the UV energy. While APO is deemed to yield higher oxidation performance, it comes also with higher costs to operate and bulkiness to the apparatus.

By way of examples of the prior art of which applicant is aware; Delta Marine International of Fort Lauderdale, Fla. U.S.A. provide for the injection of ozone into for example black water holding tanks, wherein ozone is injected into the offending tank's headspace. Also in the prior art, applicant is aware of U.S. Pat. No. 6,287,465 which issued Sep. 11, 2001 to Watanabe et al for an Ozone Deodorizer, Watanabe describing the use of an ozone generator and introducing an ozone and water mixture by an atomizer nozzle into a waste water treatment vessel. Applicant is also aware of U.S. Pat. No. 6,076,748 which issued Jun. 20, 2000 to Resch et al. for an Odor Control Atomizer Utilizing Ozone and Water, wherein the prior art is described as including an ozone generator having its output directed into a chamber containing air having organic or inorganic impurities present that are susceptible to being oxidized, the ozone being dispersed in the air to oxidize and remove odoriferous impurities. Further, applicant is aware of U.S. Pat. No. 4,654,144 which issued Mar. 31, 1987 to Sharkey et al. for a Process for the Destruction of Noxious Gases with Ozone, Sharkey et al. teaching the prior art as including a direct injection method wherein ozone is injected directly into odor-laden gas and contacted therewith using a suitable gas-gas contacter and a wet oxidation process involving contacting the odor-laden gases with ozone in the presence of water.

In the present invention, emphasis is placed upon the practical mode of delivery for photo-oxidizing technologies. Heretofore the preferred approach has been to process a contaminated air stream as it passes through an array of UV lamps and/or its ancillary equipment, and then to vent the processed air into the ambient atmosphere. One problem with this approach is the short exposure time of the pollutants to the UV photons, that is, the brevity of contact time given the photons during which to attack the pollutant molecules. A related problem arises in cases of high pollutant concentrations, where the large amass of pollutant molecules may result in some pollutant molecules being shielded from the UV photons. In both instances contaminants are left untreated, necessitating an increase in the number of UV lamps.

Thus there exists in the prior art a need for, and it is an object of the present invention to provide, an improved mode of delivery, including a method and apparatus for delivery of air processed by photo-oxidation technologies that include a UV light array.

### SUMMARY OF THE INVENTION

The method of odor control through air-facilitated injection of hydroxyl radicals of the present invention includes an array of UV radiation emitter lamps. The number of lamps in the array may vary. The lamps may be of either low- or medium pressure specification and mounted into a flow-through rigid housing. The housing may be open-ended at opposite ends. Non-polluted air is forced or drawn through an array of UV radiation emitter lamps mounted within a housing. For example, the lamp array within the housing may be aligned so that a longitudinal flow vector of air passing through the array is substantially parallel to a longitudinal axis of the housing passing orthogonally through the open ends of the housing, and directed outwardly through an outlet end of the open ends of the housing.

The air flow volume and flow rate of the non-polluted, for example outside ambient air is controlled, for example, by a fan or motorized impeller installed at, or cooperating with, one of the open ends of the housing, either pushing or drawing air through the lamp array, at an air velocity and displacement to be determined in accordance with the requirements of the application. The air being pushed or drawn is preferably non-polluted or otherwise uncontaminated ambient air from outside the confines of the chamber, space or premises being decontaminated (hereinafter “fresh air”). The outlet aperture, for example in the combination of lamp housing and fan or motorized impeller, is directed so as to point or urge the corresponding air flow vector of UV processed fresh air into a conduit directing the UV processed fresh air flow into the chamber, space or premises containing contaminated air. The conduit may be a tube or hose, of any suitable material not subject to oxidation by the UV processed air, and which is positionable relative to the fan or housing outlet, or mountable to the same.

The chamber, space or premises may be erected or constructed, or existing closed structures already in place may be used to provide a physically closed working environment or treating chamber. Such structures may be made of plastic sheet, canvas, masonry or any other material, and may be of a temporary or more permanent nature, as the need dictates. The UV unit or units (a unit being a housing containing a UV lamp or array) are located so as to discharge into the treating chamber the air stream laden with the output produced by the UV lamp array. This creates processing zones inside the closed environment downstream from the UV lamps. In certain embodiments, the supplementary use of oxidants such as hydrogen peroxide and/or catalysts such as titanium dioxide may be added. For maintenance of the lamp array, the housing may include an automated washing system for the lamps, although this is not intended to be limiting.

Thus as may be understood by one skilled in the art, the present invention provides for the generation of hydroxyl radicals in an airflow which is being inducted or driven into a workspace which may continue to be humanly habitable during the odor control and elimination process where, according to the process of the present invention, fresh air is inducted or forced through a housing containing ultraviolet lamps so as to generate hydroxyl radicals in the fresh air stream as the fresh air stream passes through an array of such lamps, the UV processed air stream then flowing downstream through a conduit which is directed into the enclosed odor containing work environment such as a room or plant or chamber which might be found in many common commercial and industrial plants such as waste treatment plants, meat rendering plants and so on where on-going odor control without the need to remove the human workers from the environment while treatment is on-going, would be advantageous.

The present invention includes a method of decontaminating air contained within an enclosed workspace. The method includes the steps of generating hydroxyl radicals in an airflow of non-contaminated air then urging the airflow into the workspace after the generating of the hydroxyl radicals in the airflow.

The method may further comprise providing a housing containing ultraviolet lamps and means for motivating the airflow through the housing so as to generate hydroxyl radicals in the airflow as the airflow passes through the housing. The lamps may be an array of such lamps, and the method may further include providing a downstream conduit in fluid communication between the housing and the workspace, and flowing the air flow downstream through the conduit so as to direct the airflow into the workspace.

In summary, the apparatus according to the present invention is for decontaminating air within an enclosed workspace located downstream and in fluid communication with the apparatus. The apparatus includes a housing containing an array of ultraviolet lamps mounted within an enclosure in the housing. The enclosure has an intake aperture and an exhaust aperture. The housing and the array form an airflow processor such that uncontaminated air entering the intake aperture passes through the array before exiting the exhaust aperture. An airflow motivator, which maybe a fan, urges the airflow through the housing and the array from the intake aperture and out through the exhaust aperture. A downstream conduit, which may be a flexible hose, is in fluid communication between the exhaust aperture and the workplace directs the airflow into the workplace after being processed in the airflow processor. The intake aperture is positionable relative to the workspace so that the airflow entering the intake aperture is uncontaminated air.

The apparatus may further include an intake conduit, which may include a rigid duct, having an upstream end exposed to ambient air external to the workspace, and an opposite downstream end mounted to the intake aperture in fluid communication with the array, wherein the array may be a parallel array which may be a vertical array of tubes.

The array of tubes may be an array including a plurality of rows of ultraviolet lamps which may be stick lamps, where adjacent rows are offset relative to one another in the direction of the airflow.

### BRIEF DESCRIPTION OF THE DRAWINGS

FIG. 1 is one embodiment of an odor control apparatus for air injection of hydroxyl radicals according to the present invention.

FIG. 2 is, in partially exploded perspective view, an ultraviolet lamp housing according to the present invention.

FIG. 3 is, in partially exploded perspective view, an alternative embodiment of the apparatus of FIG. 2.

FIG. 4 is, in unexploded perspective view, the apparatus of FIG. 3.

FIG. 5 is a sectional view along line 5-5 in FIG. 4.

### DETAILED DESCRIPTION OF EMBODIMENTS OF THE INVENTION

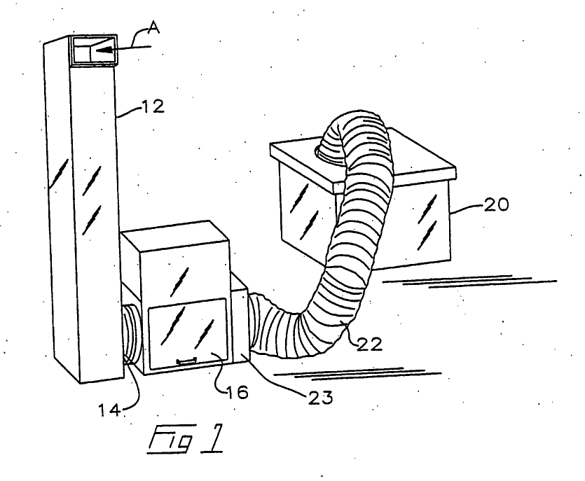

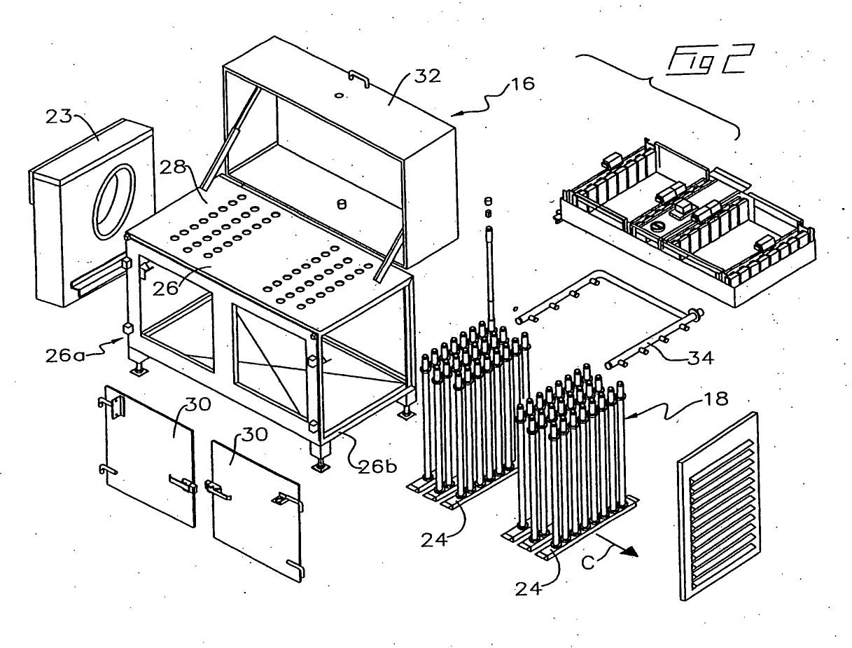

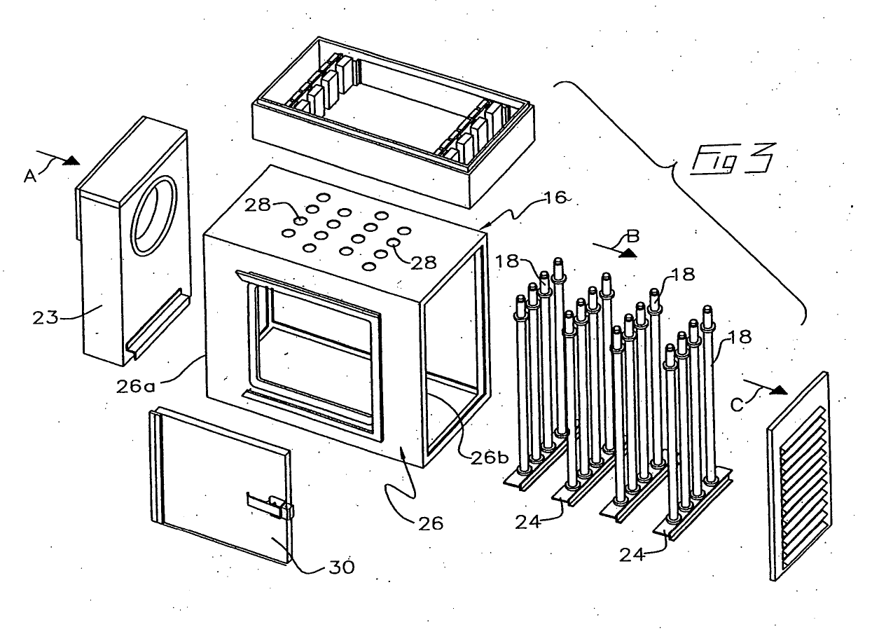

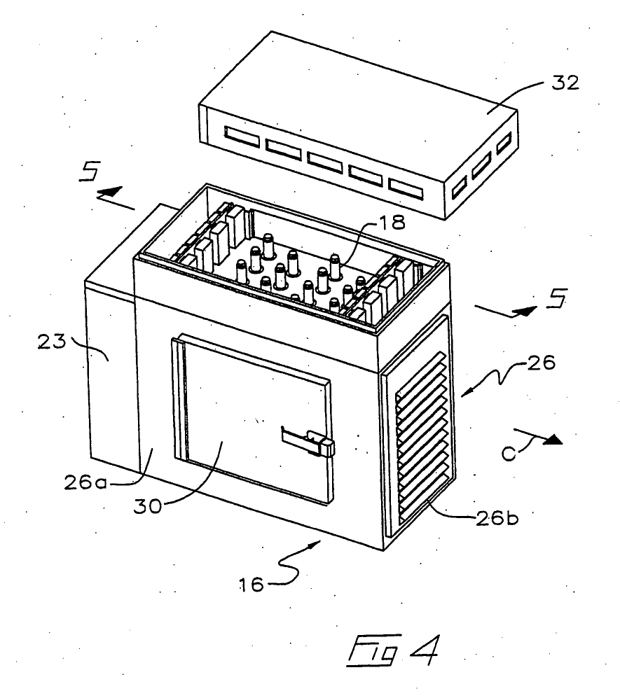

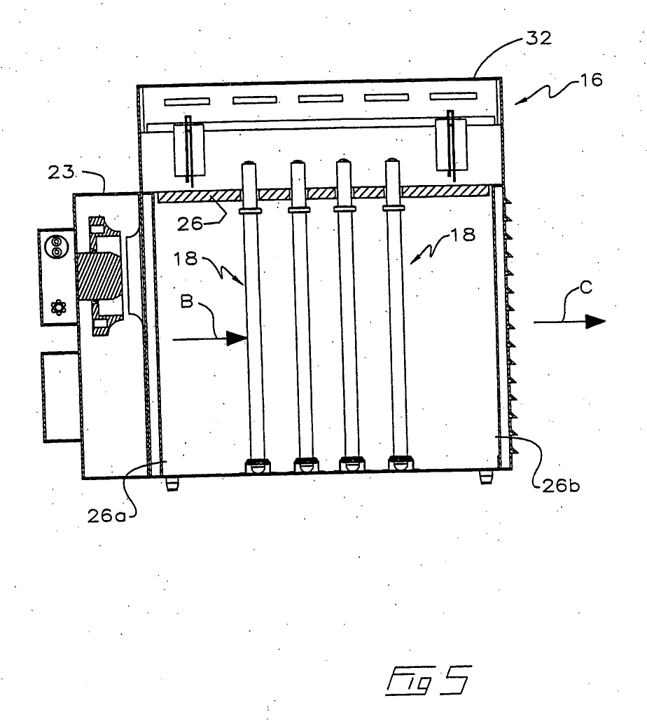

In one embodiment which is not intended to be limiting but, rather, is intended to be illustrative of the method and apparatus according to the present invention, as seen in FIG. 1, uncontaminated ambient or so-called fresh air is drawn in direction A into an intake duct 12 and passes via conduit 14 into UV lamp housing 16. As better seen in FIGS. 2-6, fresh air from intake 12 passes in direction B through an array of vertically mounted ultraviolet stick lamps 18. The air passes through an entry aperture in the side of the housing to which duct 14 is mounted. Air passes around tubes 18. The air flow may be as a result of the urging of an inductor fan already present in the well head 20 into which the distal end of outflow flexible ducting 22 is placed, or due to the operation of a fan 23 mounted in the airflow path. In particular, the upstream end of ducting 22 is mounted to the outflow aperture on the downstream side of housing 16 so that the UV processed air stream exiting the array of tubes 18 in direction C flow through ducting 22 so as to exit from the downstream end of ducting 22 into the open well head 20. The fan, whether fan 23 or the fan within well head 20, urges air through the array of lamps 18, along ducting 22, so as to draw the UV processed air stream down into the odoriferous well. Fan 23 may be contained for example in housing 16 or in intake duct 12 or the like.

Within housing 16, vertical arrays of UV stick lamps 18 are vertically mounted on lamp rack assemblies 24 and the rack lamp assemblies 24 mounted within housing 16 in spaced apart array in rows, which offset individual stick lamps between adjacent rows in the direction of airflow.

Housing 16 may include a frame 26 into which may be mounted racks 24 so as to dispose the upper ends of stick lamps 18 through apertures 28. The front of the frame may be closed by doors 30 and the top of the frame may be closed by a pivotally mounted lid or cover 32. Ducting 14 is mounted to the inlet side 26a of frame 26 and ducting 22 is mounted to the outlet side 26b of frame 26.

In the embodiment illustrated an optional spray-down system 34 may be mounted within housing 26 so as to clean lamps 18.

As will be apparent to those skilled in the art in the light of the foregoing disclosure, many alterations and modifications are possible in the practise of this invention without departing from the spirit or scope thereof. Accordingly, the scope of the invention is to be construed in accordance with the substance defined by the following claims.

What is claimed is:

1. An apparatus for decontaminating air within an enclosed workspace located downstream and in fluid communication with the apparatus, the apparatus comprising:
(a) a housing containing an array of ultraviolet lamps mounted within an enclosure in said housing, said enclosure having an intake aperture and an exhaust aperture, said housing and said array forming an airflow processor such that uncontaminated air entering said intake aperture passes through said array before exiting said exhaust aperture,
(b) an airflow motivator urging said airflow through said housing and said array from said intake aperture and out through said exhaust aperture,
(c) a downstream conduit in fluid communication between said exhaust aperture and said workplace for directing said airflow into said workplace after being processed in said airflow processor, wherein said intake aperture is positionable relative to said workspace so that said airflow entering said intake aperture is uncontaminated air.

2. The apparatus of claim 1 further comprising an intake conduit having an upstream end exposed to ambient air external to said workspace and an opposite downstream end mounted to said intake aperture in fluid communication with said array.

3. The apparatus of claim 1 wherein said array is a parallel array.

4. The apparatus of claim 3 wherein said parallel array is vertical.

5. The apparatus of claim 1 wherein said downstream conduit is a flexible hose.

6. The apparatus of claim 2 wherein said intake conduit includes a rigid duct.

7. The apparatus of claim 6 wherein said rigid duct is vertical.

8. The apparatus of claim 2 wherein said downstream conduit is flexible.

9. The apparatus of claim 1 wherein said airflow motivator is a fan.

10. The apparatus of claim 1 wherein said array has a plurality of rows of ultraviolet lamps and wherein adjacent rows of said plurality of rows are offset relative to one another in the direction of said airflow.

11. The apparatus of claim 1 wherein said array is comprised of a vertically parallel array of stick lamps.

12. The apparatus of claim 11 wherein said array includes a plurality of rows and wherein adjacent rows of said plurality of rows are offset in a direction of said airflow so as to maximize exposure of said airflow to ultraviolet radiation from said array.

13. A method of decontaminating air contained within an enclosed workspace comprising the steps of.
(a) generating hydroxyl radicals in an airflow of non-contaminated air; and,
(b) urging said airflow into said workspace after said generating of said hydroxyl radicals in said airflow.

14. The method of claim 13 further comprising providing a housing containing ultraviolet lamps and motivating said airflow through said housing so as to generate hydroxyl radicals in said airflow as said airflow passes through said housing.

15. The method of claim 14 wherein said lamps are an array of such lamps, said method further comprising providing a downstream conduit in fluid communication between said housing and said workspace, and flowing said air flow downstream through said conduit so as to direct said airflow into said workspace.
